(19) United States
(12) Patent Application Publication
(10) Pub. No.: US 2017/0067659 A1
(43) Pub. Date: Mar. 9, 2017

(54) NESTED DUCT AIR CIRCULATION SYSTEM

(71) Applicant: Michael F Silver, North Bennington, VT (US)

(72) Inventors: Michael F. Silver, North Bennington, VT (US); Merritt P. Bell, Wells, VT (US)

(73) Assignee: Michael F Silver, North Bennington, VT (US)

(21) Appl. No.: 14/846,292

(22) Filed: Sep. 4, 2015

(51) Int. Cl. F24F 7/08; A61L 9/20; B23P 19/00; B01D 46/00; F24F 13/28; F24F 13/20

(52) U.S. Cl. CPC F24F 7/08; F24F 13/28; F24F 13/20; B23P 19/00; B01D 46/0039; B01D 46/0032; B01D 46/0036; B01D 46/0086; A61L 9/20; F24F 2221/02; F24F 2221/14

(57) ABSTRACT

An air circulation and conditioning system for a building in which the supply and return ducts are coincident and vent to a room so as to form air circulation in the room including an upward return air current surrounded by a downward supply current. An array of vents may be provided, each with a return duct opening and supply duct opening to create multiple air currents in the room. A wireway may be provided for co-locating electronics and communications network along the ductwork. A modular design air handling unit with multiple compatible modules providing different air treatment, purification, and monitoring functions may be provided. A method for retrofitting an existing single duct system into a dual duct system is provided.

### TECHNICAL FIELD

The present invention relates to methods and systems for circulating and treating air in a building.

### BACKGROUND OF THE INVENTION

Typical heating, ventilating and air condition (HVAC) systems use supply ducts to supply air to a room and separate return ducts to draw air from the room. The ducts are typically connected to an air handling unit containing a motive source of circulation such as a blower. The supply and return ducts typically run along different routes. The supply and return ducts typically vent to the room at a relatively large distance away from one another. Typically, HVAC duct networks serve a singular purpose of conducting air flow. In addition, typical systems are thermodynamically inefficient and have inadequate air purification capability.

There is a need for an HVAC system in which the supply and return duct runs are coincident and vent to the room at a common location; in which a return opening may be provided within every supply opening in a room; in which electronics and communications networks are co-located with the ductwork; and in which heating, air conditioning and purification can be performed more efficiently and effectively.

The present invention fills these and other needs.

### SUMMARY OF THE INVENTION

In a first aspect, the present invention provides an apparatus for supplying air to a room in a building and returning air from the room to a motive source of air circulation, comprising: supply ductwork for carrying a flow of supply air from the source to the room; return ductwork for carrying a flow of return air from the room; at least one supply opening in the supply ductwork, said supply opening at an elevation above the floor of the room and laterally inward from the walls of the room for allowing passage of air from the supply ductwork in a downward direction to the room; and at least one return opening in the return ductwork, said return opening at an elevation above the floor of the room and laterally inward from the walls of the room for allowing passage of air from the room in an upward direction to the return ductwork; wherein at least one of said at least one return opening is centrally disposed to at least one of said at least one supply opening so that a flow of supply air and flow of return air will cause an air flow pattern within the room in which supply air flows downward from the supply ductwork and return air flows upward to the return ductwork interior of the downward supply flow.

In a second aspect, the present invention provides a method of constructing an apparatus for supplying air to a room in a building and returning air from the room to a motive source of air circulation, comprising: connecting a first end of supply air ductwork to the downstream side of the motive source; connecting a first end of return air ductwork to the upstream side of the motive source; merging the supply air ductwork with the return air ductwork so that the return air ductwork is disposed interior to the supply air ductwork downstream of the merger in the direction of the supply flow; and placing a second end of said supply air ductwork in fluid communication with the air in a room; and placing a second end of said return air ductwork in fluid communication with the air in said room interior to the second end of said supply ductwork.

In a third aspect, the present invention provides a method of providing a flow of circulating air to a room in a building and returning air from the room to the motive source of circulation, comprising: connecting a first end of supply air ductwork to the downstream side of the motive source for circulating air through the system; connecting a first end of return air ductwork to the upstream side of said source; placing a second end of said supply air ductwork in fluid communication with the air in a room at an elevation above the floor of the room and laterally inward from the walls of the room; placing a second end of said return air ductwork in fluid communication with the air in a room interior to the second end of said supply air ductwork at an elevation above the floor of the room and laterally inward from the walls of the room; activating the motive source to draw air from the room upward through the second end of the return ductwork and force air downward through the second end of the supply air ductwork around the second end of the return ductwork.

In a fourth aspect, the present invention provides a method of circulating air in a room in a building and returning air from the room to the motive source of circulation, comprising: forcing supply air through ductwork into the room through at least one supply opening in the ductwork so as to induce a downward supply current of air in the room below said opening; and concurrently with said forcing step, drawing return air through ductwork out of the room through at least one return opening in the ductwork located centrally to the at least one supply opening so as to induce a return current of air in an upward direction central to the downward supply current.

In a fifth aspect, the present invention provides a method of converting an existing supply duct of an air circulation system in a building into a dual duct, comprising: providing a return duct of small enough cross section to fit into the existing supply duct; and sliding the return duct inside the supply duct so as to form an annular space between the ducts for carrying a flow of supply air. The method may further comprise connecting the portion of the return duct that is furthest upstream in the supply duct flow direction to the upstream side of the motive source of air circulation. An opening in the supply duct is provided and connecting ductwork is disposed through said opening to connect the return duct and motive source.

### BRIEF DESCRIPTION OF THE DRAWINGS

The subject matter that is regarded as the invention is particularly pointed out and distinctly claimed in the concluding portion of the specification. The invention, however, may be best understood by reference to the following detailed description of various embodiments and the accompanying drawings in which:

FIG. 1 is an elevation view of an air circulation system of the present invention.

FIG. 2 is a perspective view of the air circulation system of FIG. 1.

FIG. 3A is a side view of a convergence of return and supply ductwork of the present invention.

FIG. 3B is a top view of the convergence of FIG. 3A.

FIG. 3C is a close up view of the convergence of FIG. 3B.

FIG. 4A is a perspective view of a compact convergence of the present invention.

FIG. 4B is another perspective view of a compact convergence of FIG. 4A.

FIG. 4C is a side view of a compact convergence of FIG. 4A.

FIG. 4D is a bottom view of a compact convergence of FIG. 4A.

FIG. 5 is a perspective view of an assembly of a compact convergence of FIG. 4A and a diffuser of the present invention.

FIG. 6 is a cutaway perspective view of a plenum and branch ductwork of the present invention.

FIG. 7A is a perspective view of a flow adjuster of the present invention.

FIG. 7B is a front view of FIG. 7A.

FIG. 7C is a right side view of FIG. 7B.

FIG. 7D is plan view of a flow adjuster blade of FIG. 7B.

FIG. 7E is a plan view of another flow adjuster blade of FIG. 7B.

FIG. 8 is a perspective view of a diffuser of the present invention from the bottom.

FIG. 9 is a perspective view of the diffuser of FIG. 8 from the top.

FIG. 10A is a perspective view of the diffuser of FIG. 8 from the bottom with the cover hinged open.

FIG. 10B is a perspective view of the diffuser of FIG. 8 from the top with the cover hinged open.

FIG. 11 is a perspective view of another diffuser of the present invention from the bottom.

FIG. 12A is a perspective view of a wireway of the present invention with an open channel cover.

FIG. 12B is an end view of the wireway of FIG. 12A with a closed channel cover.

FIG. 12C is an end view of the wireway of FIG. 12A.

FIG. 13A is another view of FIG. 12A.

FIG. 13B is a perspective view of an assembly of a wireway and ductwork of the present invention.

FIG. 14A is a perspective view of an air sample module of the present invention.

FIG. 14B is a close up perspective view of a portion of the air sample module of FIG. 14A.

FIG. 14C is a front view of FIG. 14A.

FIG. 14D is a right side view of FIG. 14C.

FIG. 15A is a perspective view of a roller spacer of the present invention.

FIG. 15B is a front view of FIG. 15A.

FIG. 16A is a perspective view of ductwork of the present invention with return ductwork partially inserted in supply ductwork and roller spacers attached to the return ductwork.

FIG. 16B is a close up view of a portion of FIG. 16A.

FIG. 17A is a perspective view of a connection between supply ductwork and return ductwork of the present invention.

FIG. 17B is a perspective view of a joint between adjacent return ducts of the present invention, with the ducts truncated.

FIG. 18A is a perspective view of an assembly of an air handling unit and ductwork of the present invention.

FIG. 18B is another perspective view of FIG. 18A.

FIG. 19A is another perspective view of FIG. 18A.

FIG. 19B is a perspective view of an air sample module of the present invention.

FIG. 19C is a perspective view of a dump valve of the present invention.

### DETAILED DESCRIPTION OF THE INVENTION

The term “downstream” herein means in the direction of the flow of air and “upstream” means in the opposite direction of flow. An “upstream” location is located a distance away from the point of reference in a direction opposite the flow direction. The “upstream side” of an object refers to the side facing the upstream direction. For example, the upstream side of a fan is the air intake side of the fan and the downstream side of a fan is the air exit side of the fan.

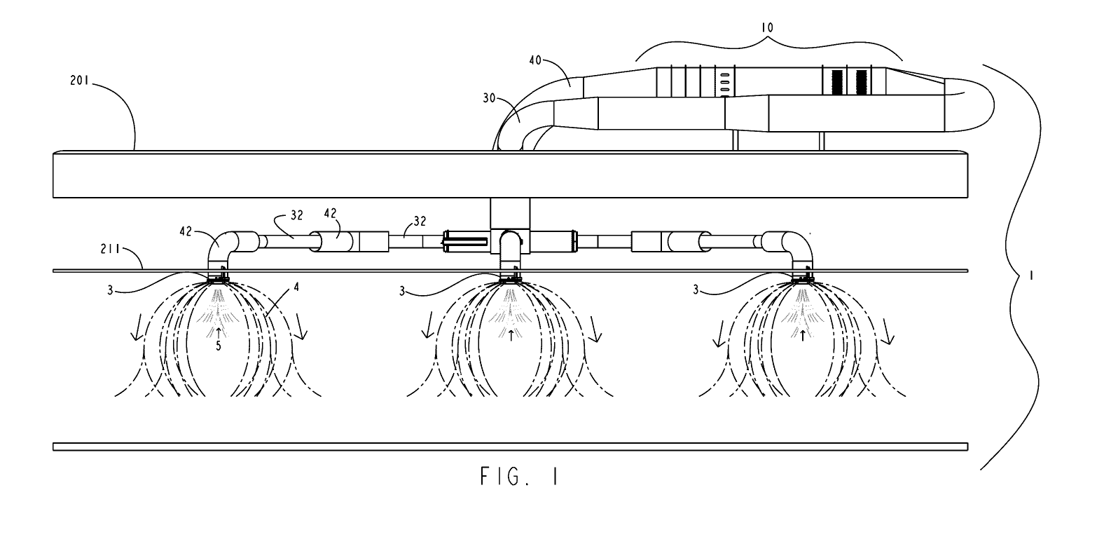

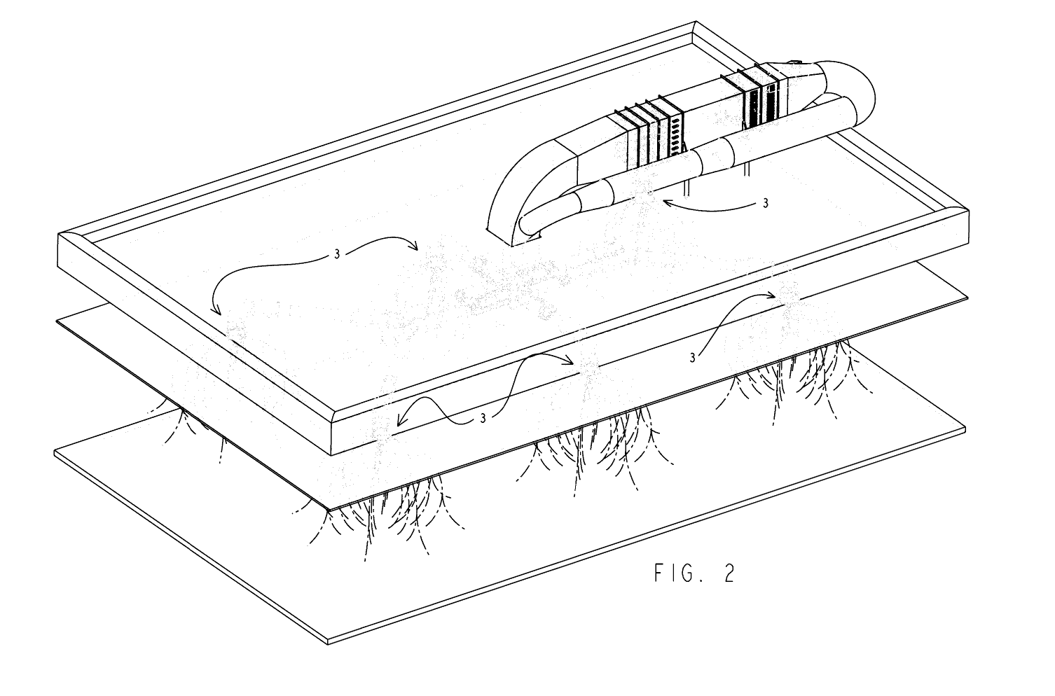

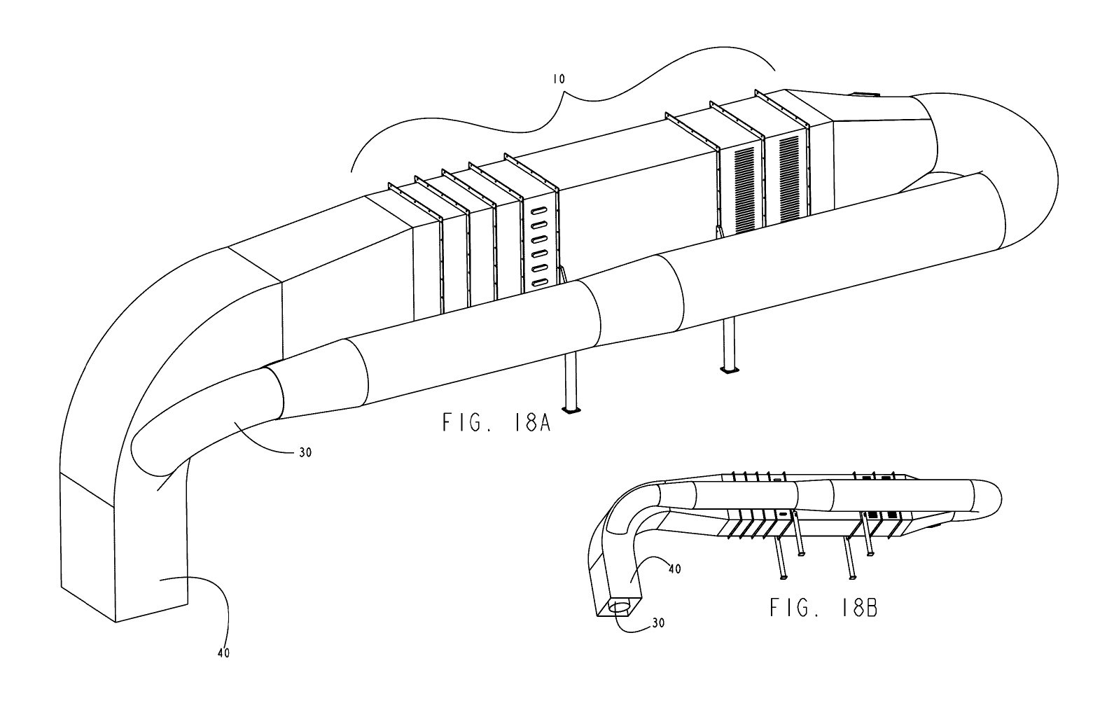

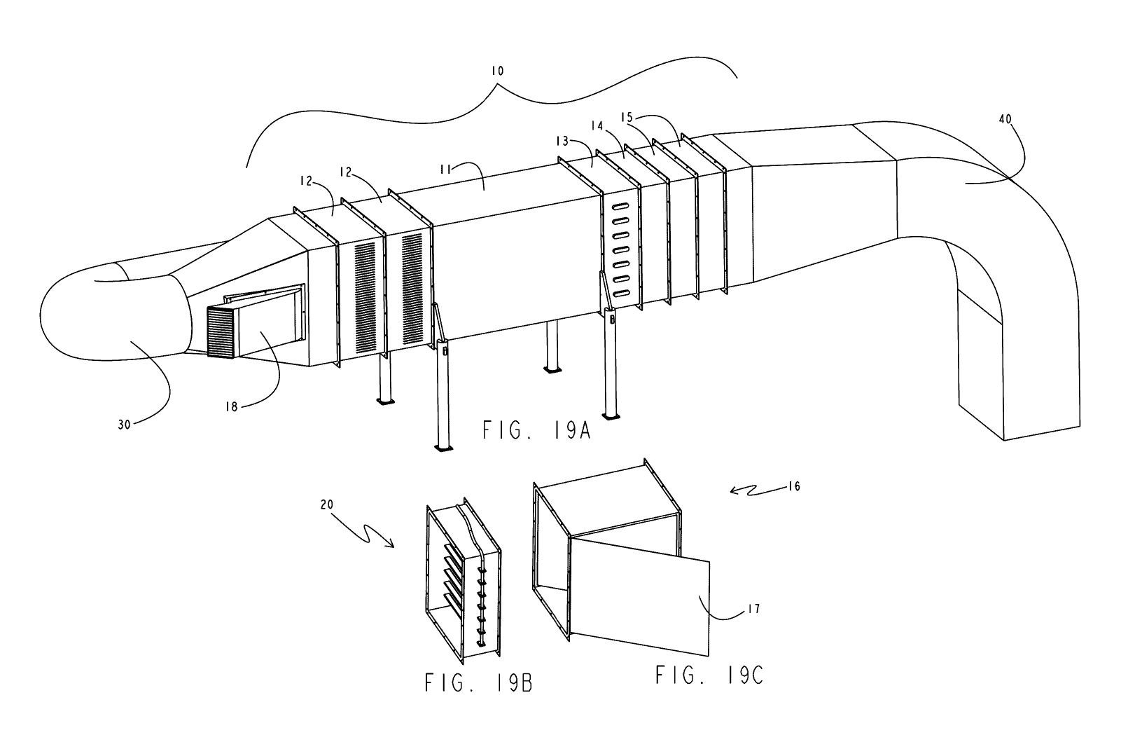

With reference to FIGS. 1 and 2, a preferred embodiment of the HVAC system 1 of the present invention may comprise an air handling unit 10, which unit comprises a motive source of circulation such as a blower 11 (FIG. 19A). The downstream side of air handling unit 10 is connected to supply ductwork 40 and the upstream side of the unit is connected to return ductwork 30. Air handling unit 10 draws air through ductwork 30 and blows the air through supply ductwork. Supply ductwork 40 carries the air to a room, and return ductwork 30 carries air from the room to unit 10. The supply and return ductwork each have at least one opening into at least one room in the building. In the embodiment shown, the supply and return ductwork merge (see FIGS. 18A and 18B) so that the return ductwork is interior to the supply ductwork so that the paths of the two ducts coincide for some distance. The main ductwork may divide into multiple branches, each of which may comprise a return branch 32 within a supply branch 42. In the embodiment shown, each branch terminates at a vent where supply air is blown into the room and return air is drawn from the room so as to form a downward supply current 4 and upward return current 5 interior of the supply current. Thus is formed an air flow circulation around a continuous path comprising air handling unit 10 connected to supply ductwork 40 connected to room 210 connected to return ductwork 30 connected to air handling unit 10.

Air handling unit 10 may be mounted on a roof 201 of the building.

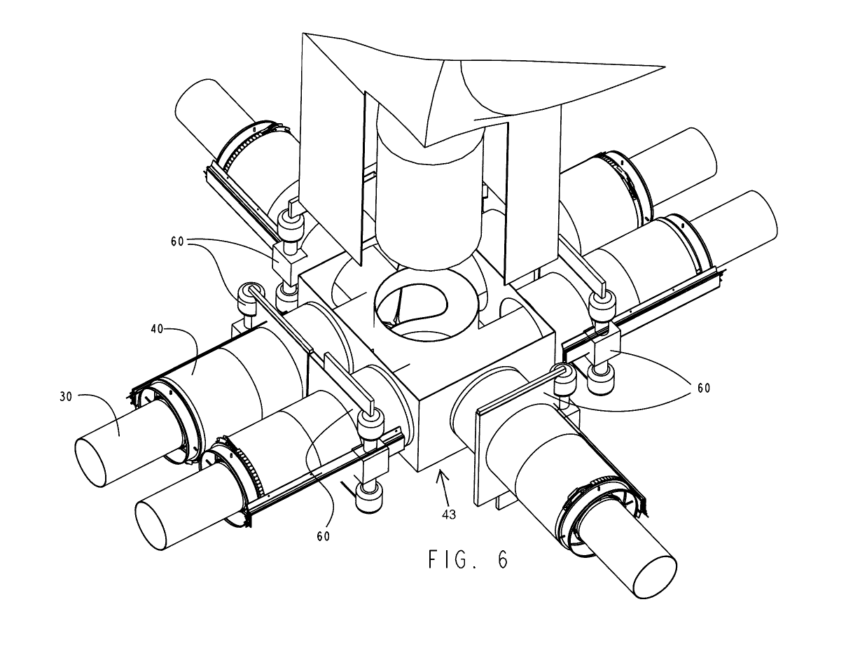

FIGS. 1 and 2 show an embodiment of the present invention with multiple branches and multiple vents where each branch vents to the room. The branches may branch off from a plenum 43 (FIG. 6). Although six are shown, there may be any number of branches and vents. The vents 3 may be spaced in a regular or irregular pattern and may be spaced in any arrangement as necessary to meet needs and requirements presented under any particular scenario, to accommodate variables such as the characteristics of the air handling unit, size and shape of the room, uses of the room, and desired flow rates.

The term “vent” in noun form refers to a locus or loci for passage of air, and not necessarily structure except structure expressly recited. A vent may comprise a supply duct opening and a return duct opening at a common location or in close enough proximity so that they may cooperate to promote a local air circulation in the room in which supply air flows downward from the vent and return air flows upward to the vent interior of the supply flow. A vent may comprise a return duct opening centrally located within a return duct opening.

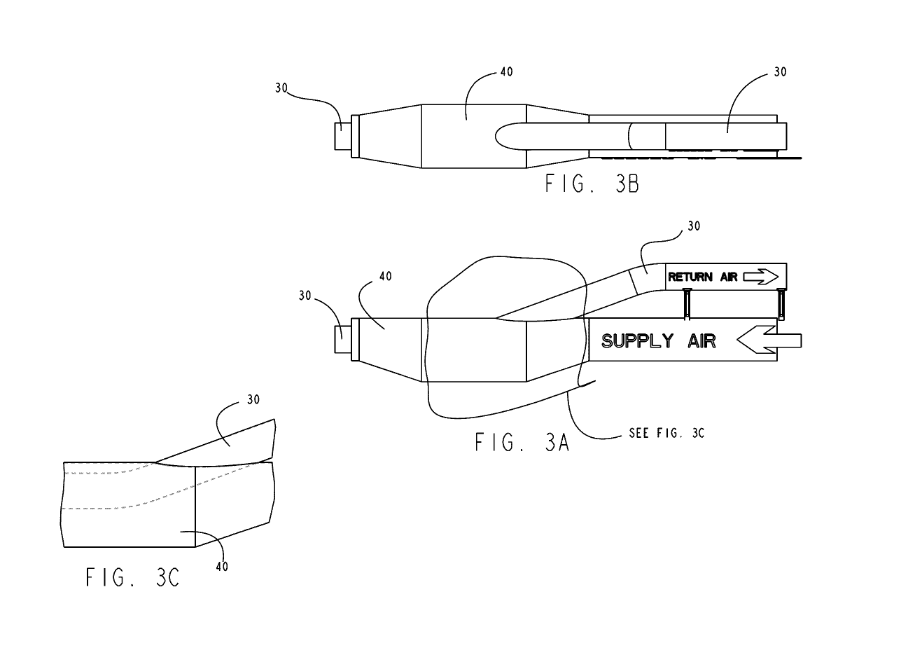

With reference to FIGS. 3A-3C, the return ductwork 30 converges with the supply ductwork 40 through an opening in the side of the supply ductwork and is disposed inside the supply ductwork downstream of the convergence in the direction of the supply flow. The circumference of the supply ductwork may expand to accommodate the return ductwork and maintain sufficient flow area.

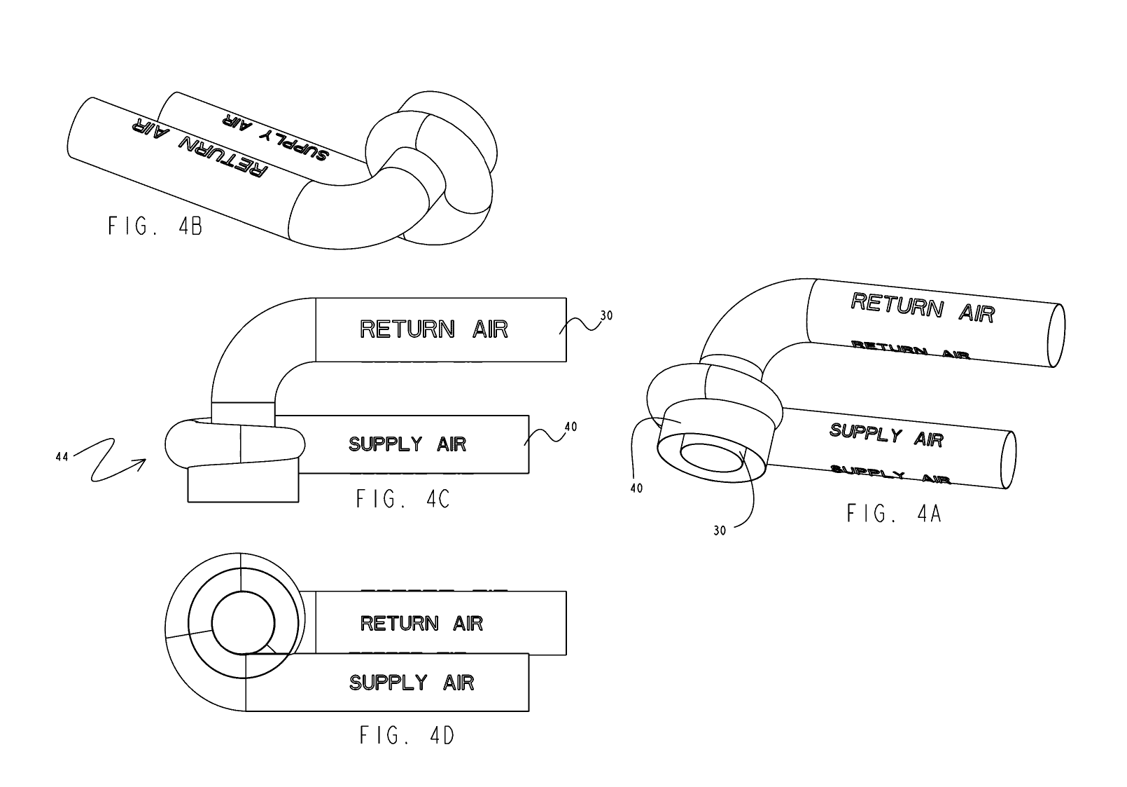

With reference to FIGS. 4A-4D, in another embodiment, the opening in the supply ductwork is formed by a coaxial transition section 44 in which the circumference of the supply ductwork expands and the longitudinal direction of the supply ductwork changes course to conform with the longitudinal direction of the return ductwork. The upstream portion of the transition section is shaped to channel the supply flow to one side of the return flow ductwork. The transition section then transitions the flow into an annular space around the return ductwork. The ducts may converge at any location along the path of the ducts as may be desired, and may diverge and re-converge.

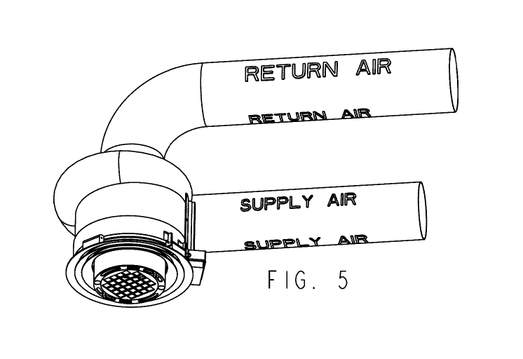

With reference to FIG. 5, the convergence may occur in close proximity to the room and may comprise a transition section connected to the diffuser. For ductwork exposed in the room, the convergence may occur in the room.

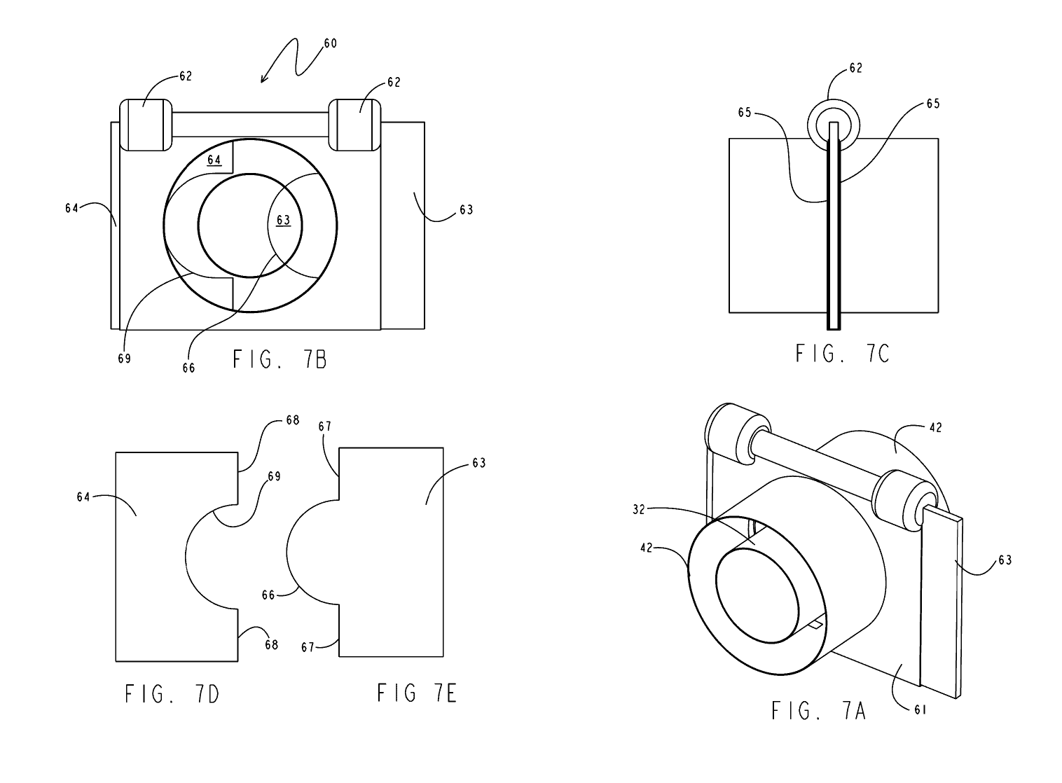

With reference to FIG. 6, each branch may be equipped with a flow adjuster 60. With reference to FIGS. 7A-7E showing a preferred embodiment, transverse slits (not shown) may be provided in the supply and return duct walls for slidably receiving blades of flow adjuster 60. The slits are a sufficient length to accommodate entry of the blades. Flow adjuster 60 may comprise a scabbard 61 attached to branch supply duct 42 for housing one or more slidable blades 63 and 64 that are slidable over a range from an open position to closed position. In the embodiment shown, the flow adjuster comprises a first blade 63 and second blade 64 that are slidable towards each other to close the ducts and away from each other to open the ducts. First blade 63 comprises a rounded front portion 66 conforming to the size and shape of the return duct's concave interior wall surface, and shoulder edges 67 for contacting the leading edges 68 of second blade 64. Second blade 64 comprises leading edges 68 and an edge 69 having a concave profile forming a pocket that conforms to the size and shape of the return duct's convex exterior wall surface.

With further reference to FIGS. 7A-7E, blades 63 and 64 are each connected to a motor 62, which is an electric stepper motor in a preferred embodiment. Motors 62 move the blades over a range of motion from an open position to a closed position. In the open position, the blades are separated from one another and retracted from the ducts. In the closed position, edges 68 of blade 64 contact edges 67 of blade 63, and concave edge 69 of blade 64 closes against the outside surface of branch return duct 32 to close off the annular passage of branch supply duct 42; and convex edge 66 of blade 63 closes against the interior surface of duct 32 to close off the return flow. Flexible membrane seals seal the slits when the blades are retracted and seal the space between the ductwork and blades when the blades are inserted. An opposing seal is attached to the ductwork on either side of each slit. The opposing seals overlap each other so that they cooperate to seal the slits.

While the ductwork of the preferred embodiment may be round, the ductwork may have other shapes, such as rectangular or other polygonal shapes. Ductwork may transition from one size or shape to another size or shape. Supply ductwork may have different shape than return ductwork. Flow adjuster blades 63 and 64 are provided to conform to the duct shape.

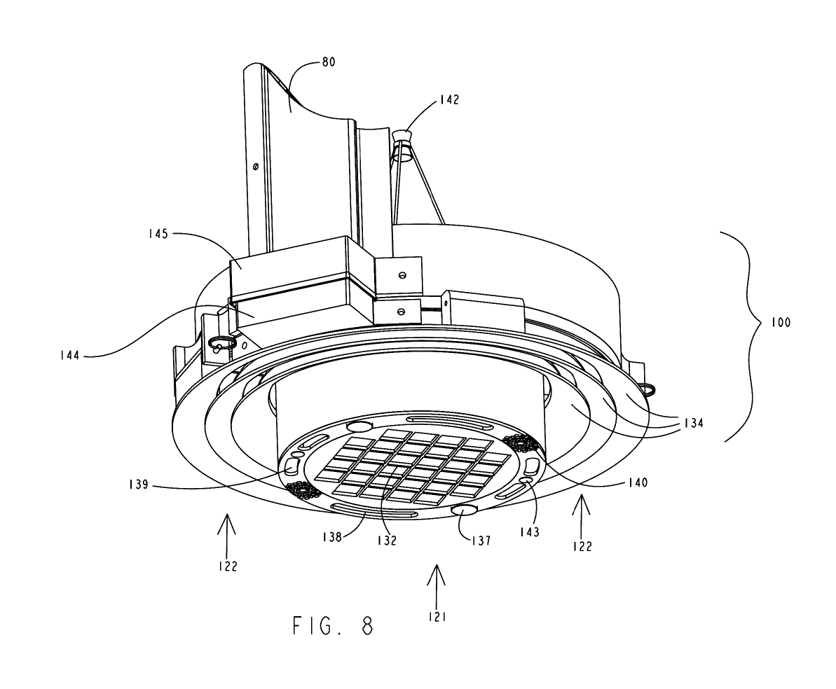

With reference to FIGS. 8, 9, 10A and 10B, one embodiment of the present invention comprises a diffuser 100 connected to the ductwork at the fluid interface between the ductwork and the room. The diffuser of FIG. 8 comprises central return portion 121 and annular supply portion 122 which correspond with central return duct and annular space of the outer supply ductwork. The term “central” does not require concentricity or symmetry between the supply and return ducts or the supply and return portions of the diffuser. For example, a return duct within a supply duct is central to the supply duct even if it is not concentric and does not have the same shape. A return duct and its opening into a room may be considered central to a supply duct and its opening into a room even if the return duct extends beyond the supply duct opening as long as it is within the periphery of the supply duct opening when viewed from directly below.

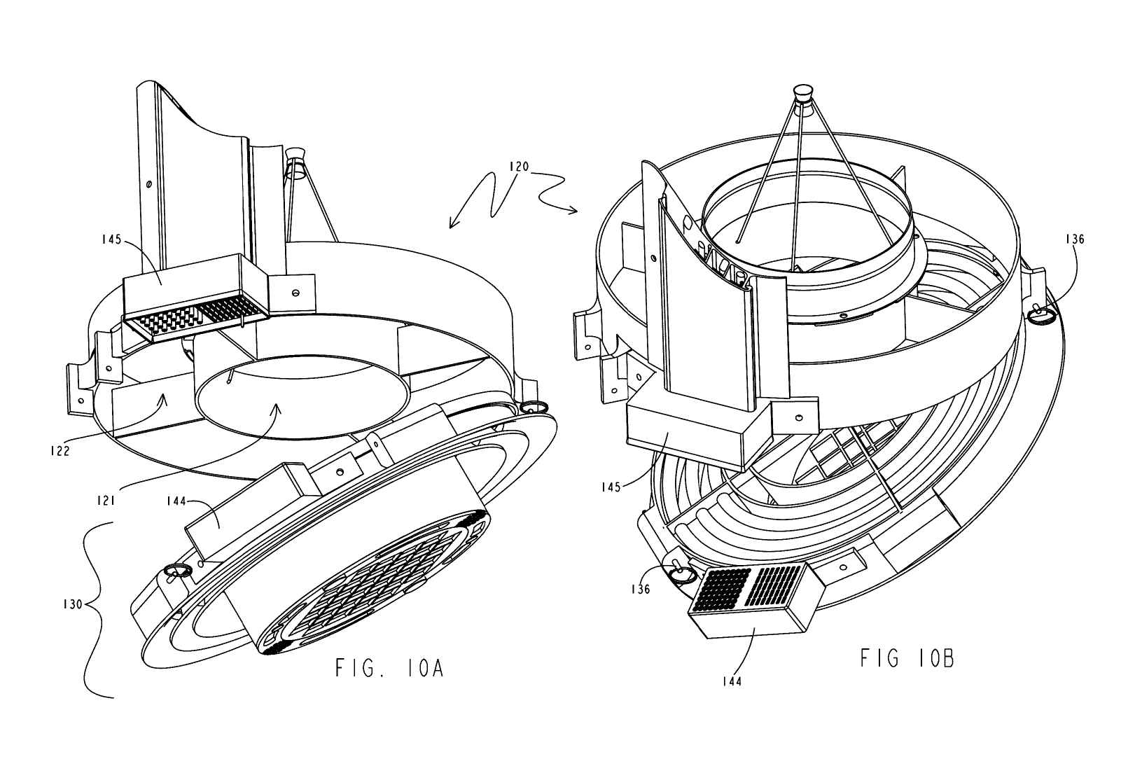

With reference to FIGS. 10A and 10B, diffuser 100 comprises a support 120 for connection to the ductwork and a cover 130 releasably attached to support 120. With reference to FIG. 8, supply portion 122 of cover 130 comprises supply louvers 134 for directing supply air flow outwardly and downwardly from the diffuser. Return portion 121 may comprise a grill 132 with an open lattice framework through which return air is drawn in an upward direction interior to the supply air flow. Supply air is blown and return air is drawn through the ductwork and diffuser of the present invention so as to form an air current comprising a downward supply current 4 (FIGS. 1 and 2) and upward return current 5 (FIGS. 1 and 2) in the room in the vicinity of the diffuser.

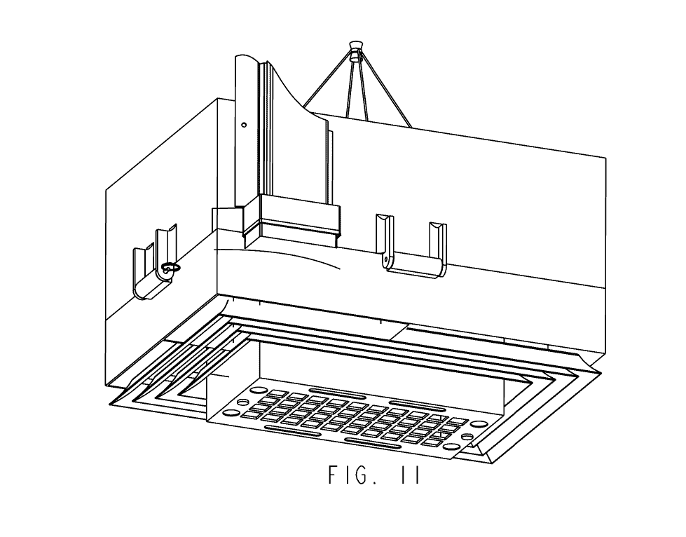

The diffuser may be round as in FIG. 8, rectangular as in FIG. 11, another shape conforming to the shape of the ductwork, or any other desirable shape.

The present invention provides many mechanical advantages and improved fluid-dynamic and thermo-dynamic performance over current systems. Coincidence of return and supply duct runs provides for more compact construction and more efficient use of building space. Significantly less insulation material is required to insulate coaxial ducts compared to separate ducts of the same length and flow area. Coincident supply and return openings provides unique adaptability and scalability to meet the needs of various room sizes and shapes. As shown in FIGS. 1 and 2, multiple supply/return vents may be dispersed throughout the room, thus creating multiple localized air currents that more efficiently circulate air throughout the room. The dispersal pattern of such vents may be tailored to room size and geometry. Non-uniform dispersal of vents may be used to provide non-uniform flow currents as may be desired for non-uniform needs within a room. Vent elevations above the floor may be variable from vent to vent to adapt to different needs in different regions of the room. Flow adjusters 60 (FIGS. 6 and 7) may be provided and separately controlled to separately control flow velocity and volumes for each branch of a multi-branch system (FIGS. 1, 2 and 6). Thus, the system of the present invention provides improved performance, flexibility, and scalability with fewer resources and greater efficiency than current systems.

The coincident supply/return vents of the present invention, each providing dual supply and return capacity, provides new HVAC system design flexibility and design methodologies. Systems may be designed in scalable, modular fashion. Design requirements may be achieved by selecting the number and placement of vents having known individual performance profiles and that cooperate together in known and scalable aggregate performance profiles.

Dispersal of multiple supply/return vents provides more effective and uniform air circulation and reduces or eliminates localized stagnation or inadequate air flows.

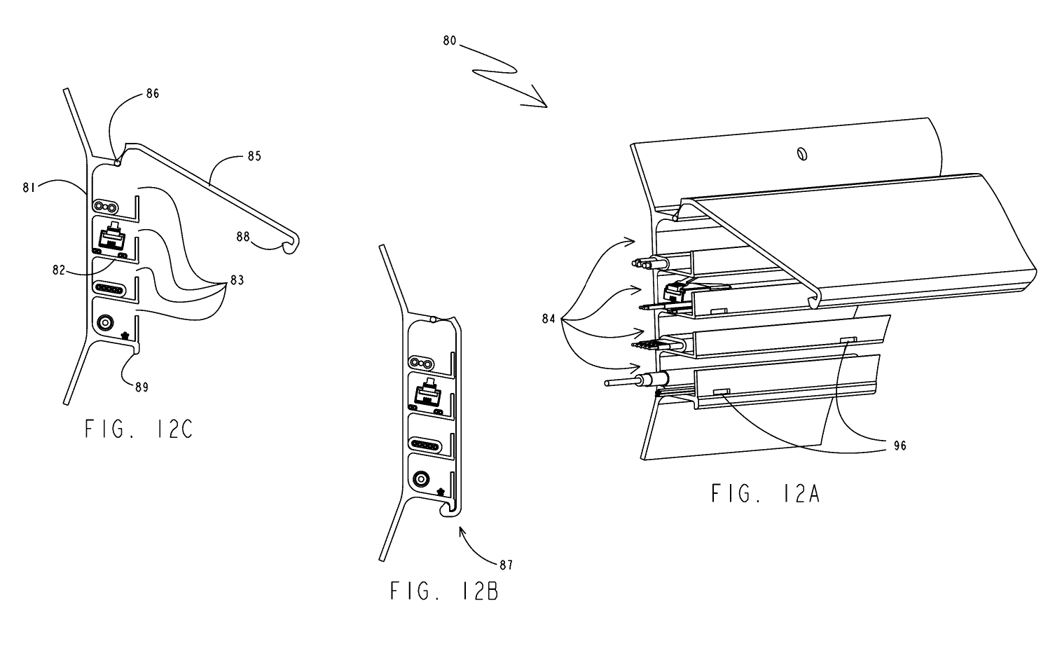

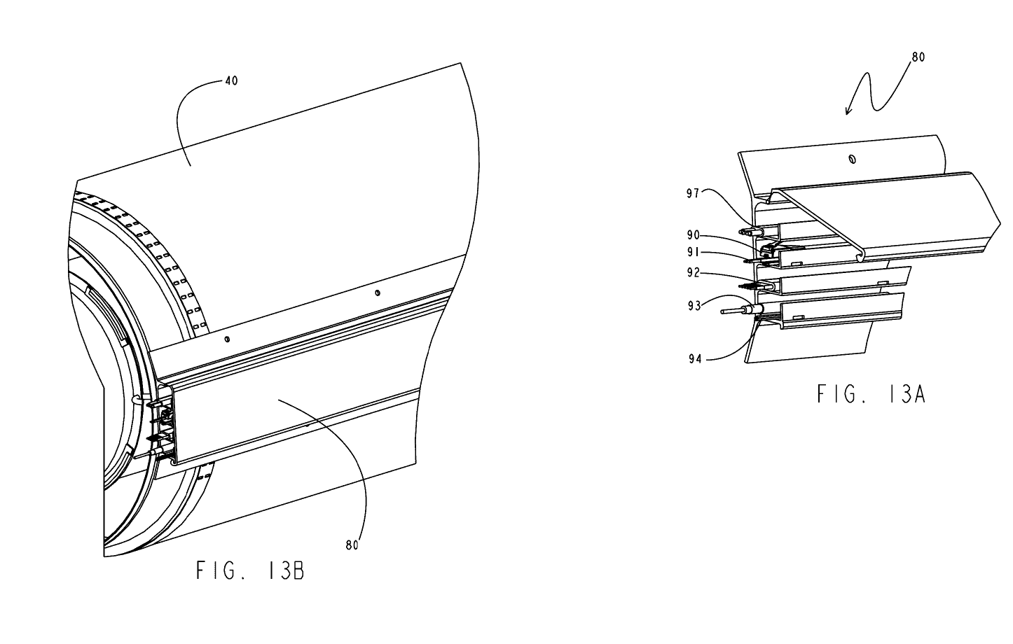

In reference to FIGS. 12A-13B, the present invention may comprise a wireway 80 that is attachable to the ductwork for carrying various types of circuitry, such as electrical wires and fiber optics. The wireway may run along the ductwork for any distance and in any path. With reference to the preferred embodiment of FIGS. 12A-12C, wireway 80 may comprise a base 81 with channel troughs 82 extending from the base forming channels for receiving wiring along the length of the wireway. Channel troughs 82 are spaced apart so as to form channel side openings 83 through which wiring may be installed and removed. Wireway 80 may be provided with channel cover 85 hingedly connected to base 81 via hinge 86, which hinge may be sealed to provide a moisture barrier. Cover 85 comprises a lip 88 for cooperating with lip 89 of the base to form a releasable snap latch connection 87 for releasably covering the channels. The channels may have open ends 84. Channel troughs 82 may have condensate vents 96 to allow escape and evaporation of condensate from the channels.

With reference to FIG. 13A, wireway 80 may carry any variety of circuitry, such as ethernet 90, audio 91, multi-conductor 92 (for video surveillance, smoke and fire detector, and motion detector), coaxial cable 93, fiber optics 94, and line voltage wire 97.

Wireway base 81 may be provided with adhesive strips as means of attachment to the ductwork. Base 81 may be provided with holes for receiving threaded fasteners or other fastener types for attachment to the ductwork.

The length of the wireway is as long as may be required by the desired application. The wireway may be provided in segments of the same or different lengths attached endwise to form long continuous runs.

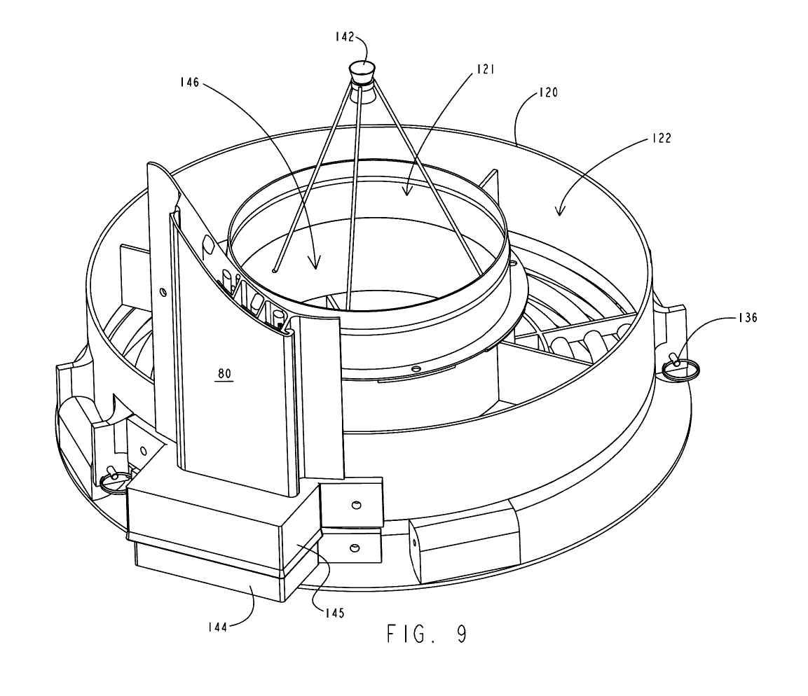

With reference to FIGS. 8 and 9, diffuser 100 may comprise an air filter (not shown) disposed in filter location 146 of the return portion 121 of support 120 above grille 132, said filter for trapping airborne dust. Diffuser may further comprise a pressure sensor 142 or flow rate sensor (not shown) attached to support 120 and disposed above filter location 146 for detecting pressure and pressure changes in the ductwork, such as changes that may be caused by a fouling air filter in location 146. Diffuser 80 may further comprise one or more of the following types of instrumentation, alone or in combination: room surveillance cameras 137; lights 138; emergency lighting; indicator lights 143 to signal pressure sensor information; audio speakers 140; air quality indicator lights; detectors and alarms for smoke, heat, carbon dioxide, carbon monoxide, and matter and conditions; and motion detectors.

Pressure sensor 142 in circuit with indicator light 143 may indicate when the air filter in location 146 of the diffuser needs to be changed or serviced.

The diffuser may comprise circuitry necessary to support the electronic, fiber optic, and other types of equipment and instrumentation and may be adapted for receiving retrofit instrumentation and circuitry. Diffuser 100 may comprise a circuitry connector 144 connected in circuit with diffuser equipment and instrumentation, said connector adapted for releasable connection to external circuitry having a compatible connector. The diffuser may further comprise compatible connector 145 connected in circuit with the circuitry carried in wireway 80. Connectors 144 and 145 may be pin and socket type connectors or any other suitable connector type. Connectors 144 and 145 may be releasably connected to accommodate repeated disconnection for maintenance and upgrades.

With reference to FIGS. 9A-10B, cover 130 is releasably attachable to support 120. Releasable attachment may be by means of one or more pinned hinge joints comprising a pin 136 releasably inserted into aligned holes in the support and cover. The cover may rotate between open and closed positions about said hinged joint. Cover 130 may be held in the closed position by another releasable pinned joint. Any other suitable means of releasable attachment between the cover and support may be used.

In the embodiment of FIGS. 8-11, cover 130 comprises connector 144 and support comprises compatible connector 145, which connectors are connected in circuit with one another when the cover is closed, and are disconnected when the cover is opened.

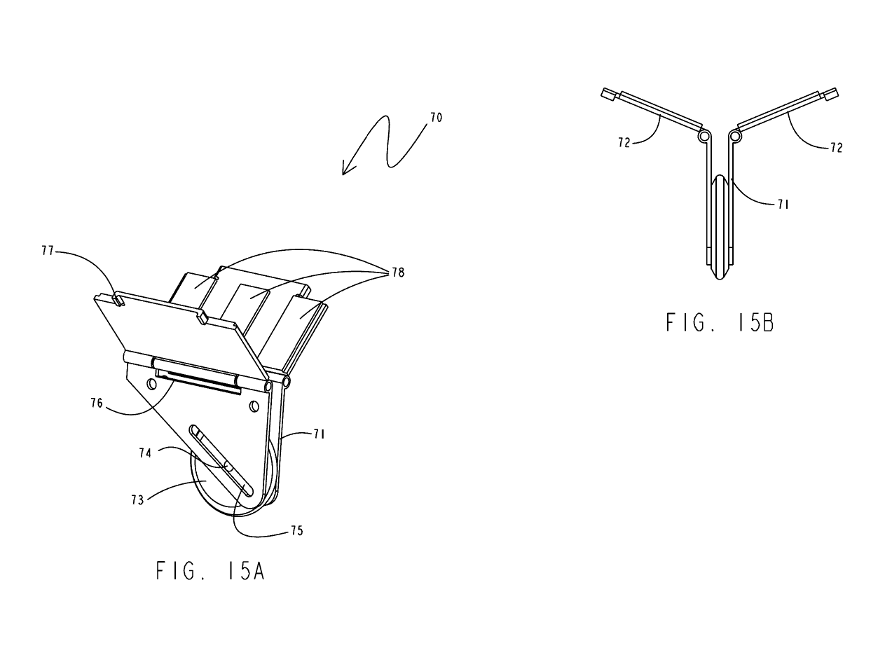

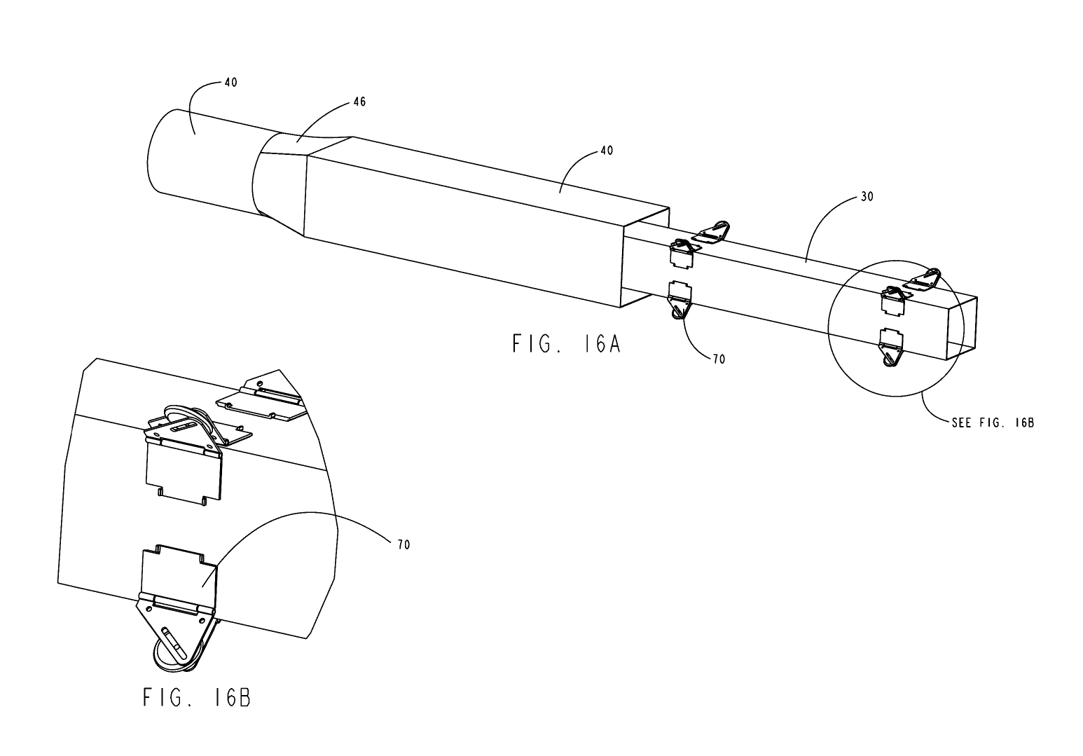

With reference to FIGS. 15A-16B, ductwork 30 and 40 may comprise roller spacers 70 for maintaining spacing and alignment of the ductwork 30 and 40 with respect to each other and for facilitating installation of ductwork 30 within ductwork 40. Roller spacers may comprise a body 71 attached to a ductwork 30 and a wheel 73 rotatably connected to the body with axel 73 and in rolling contact with ductwork 40 at the peripheral wheel surface. In an alternate embodiment, the body may attached to ductwork 40 with the wheel in rolling contact with duct 30.

FIG. 16A shows two sets of roller spacers attached to a rectangular duct 30, each set spaced apart from one another longitudinally along the duct, and each set comprising a roller at each corner of the rectangle. Any desirable number and spacing of sets may be used, and any desirable number and spacing of rollers within a set may be used as necessary to maintain spacing, stability and ease of insertion of one duct into another.

Said roller spacers simplify installation of the inner duct with the outer duct. The roller spacers also facilitate a method of introducing a new return duct to convert an existing supply duct into a dual duct. Sliding the new return duct inside the supply duct forms an annular space between the ducts for carrying supply air. The method may further comprise connecting the portion of the return duct that is furthest upstream in the supply duct flow direction to the upstream side of the motive source of air circulation. An opening in the supply duct is provided, and connecting ductwork is disposed through said opening to connect the return duct and motive source. The roller spacers maintain desired spacing for the annular flow area. Other structure may be used to provide the spacing. With the return and supply ducts thus connected to the motive source of circulation and in fluid communication with the room as described elsewhere herein, the existing system may be converted to a system of the present invention to cause air circulation in the room with a downward supply current around an upward return current.

Roller spacer 70 may comprise body 71 and flanges 72 hinged to the body, said flanges for connecting to ductwork. Body 71 may comprise at least one opening 75 for receiving a wheel axel 74. Wheel 73 is connected to body 71 via axel 73 inserted in opening 75. Opening 75 may be an elongated slot disposed at an angle to the hinges so as to provide adjustability in the axel position and overall spacer height as measured from the hinge to peripheral wheel surface. Hinged flanges permit flush attachment of the flanges onto flat surfaces or onto two adjacent surfaces forming a range of angles from acute to obtuse.

Roller flanges 72 may be provided with adhesive strips 78 for attachment to a duct. Roller body 71 may be provided with a slot 76 for receiving a band so that roller may be attached to a duct with a band clamp clamped around the duct. Roller flanges 72 may comprise tabs 77 forming a guide for receiving a band and stabilizing the roller in desired alignment with the band.

In the embodiment of FIG. 16A, ductwork 30 may be slid into ductwork 40 while roller spacers 70 maintain spacing and roll to minimize sliding force.

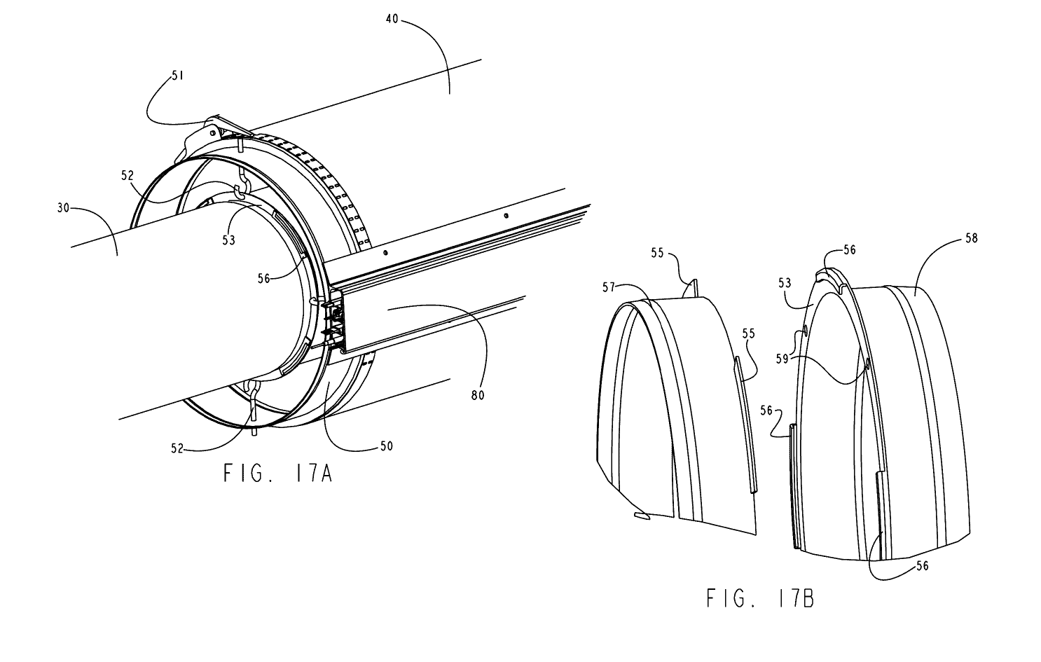

With respect to FIGS. 17A and 17B, suspension coupling 50 of short length and of roughly the same outside diameter as the inside diameter of ductwork 40 may be inserted into the end of ductwork 40 and cinched in place by band clamp 51 around ductwork 40. Suspension hooks 52 may be connected to and disposed radially inward from suspension coupling 50. Hooks 52 have a hooked distal end connected to ductwork 30.

With reference to FIG. 17B, an embodiment of the present invention provides return duct couplings 57 and 58 attached to the abutting ends of return duct segments for connecting said segments together endwise. Coupling 57 comprises one or more tabs 55 extending radially outward, which tabs are disposed in one or more corresponding slots 56 of coupling 58. Slots 56 are formed on a flange 53 of coupling 58. Assembly comprises attaching the end couplings to a first and second return duct segment, bringing the adjacent ends of the first and second segments together so that tabs 55 abut against flange, and rotating one or both duct segments to dispose tabs 55 into slots 56. The connection may be releasable by reverse rotation. The couplings may be inserted into the ends of the return duct segments and may have an interference fit with the inside diameters of said segments. Any suitable means of affixation of the couplings to the return duct segments may be used.

Flange 53 comprises holes 59 for receiving hooks 52. In the embodiment of FIG. 17A, hooks 52 comprise four hooks equally spaced apart around the ductwork. Thus, ductwork 30 and 40 are connected together and spaced apart by hooks 52.

With reference to FIGS. 18A, 18B and 19A, air handling unit 10 comprises a blower 11 or other motive source of air circulation (also referred to herein as “circulator”). Supply ductwork 40 is connected to air handling unit 10 on the downstream side of said unit, and return ductwork 30 is connected to the upstream side of the air handling unit. Air handling unit may further comprise one or more dehumidifiers (not shown), electrostatic precipitators 12, ultraviolet germicidal irradiators 13, HEPA/MERV filtration modules 14, and carbon filter modules 15. In a preferred embodiment, electrostatic precipitators 12 are upstream of circulator 11, ultraviolet germicidal irradiators 13 are downstream of circulator 11, HEPA/MERV filtration modules 14 are downstream of the germicidal irradiators 13, and the carbon filtration modules 15 are downstream of the germicidal irradiators 13. The various modules may be configured with compatible size, shape and connecting structure so that they may be readily assembled in various combinations and sequences and multiple modules may be stacked in any combination or number. Such modular design also promotes simple and fast replacement and maintenance of modules.

Electrostatic precipitators 12 may comprise charged aluminum plates for electrostatically precipitating matter out of the air stream, including particulate matter ranging in size from 0.01 to 1 micron.

Ultraviolet germicidal irradiators 13 may comprise ultraviolet light sources which emit short wave ultraviolet radiation, which is known to kill or disable bacteria, viruses, molds, and other microorganisms and pathogens.

HEPA/MERV filtration modules 14 comprise a High Efficiency Particulate Arrestance (“HEPA”) filter or an air filters having a Minimum Efficiency Reporting Value (“MERV”) in the range of 13-20, or a combination thereof. HEPA filters remove at least 99.97% of particles that have a size of 0.3 microns from the air passing through the filter. Filters in the MERV 13-20 range are effective at filtering particulate of 1 micron in size and smaller.

Carbon filter modules 15 comprise activated carbon filters for absorbing odors, vapors and other hydrocarbons and chemicals.

In a preferred embodiment, HEPA/MERV filtration modules 14 are adjacent to and downstream of germicidal irradiators 13. In this embodiment, irradiators 13 direct ultraviolet radiation at contaminants in the air flow within the irradiator and at the upstream side of the adjacent HEPA/MERV filters. Contaminants trapped on the filters may be exposed longer to ultraviolet radiation, which enhances the effectiveness of the radiation.

A fresh air intake 18 may be connected on the upstream side of the air handling unit. Intake 18 may be opened as needed to permit intake of atmospheric air into the circulation as needed.

With reference to FIG. 19C, a dump valve 16 may be connected to air handling unit 10 to divert contaminated air out of the building and draw atmospheric air in. In a preferred embodiment, dump valve 16 is connected upstream of blower 11. Dump valve 16 is of compatible size and shape to fit with the adjacent ductwork or air handling unit components. In a preferred embodiment, dump valve 16 is rectangular with opposing side walls 17 that are hingedly mounted on their upstream side so that they may swing in synchronized fashion from a closed position to an open position. In the closed position, the four walls form a strait rectangular duct having a rectangular flow channel for uninterrupted through-flow. In the opened position, side walls 17 are swung in the same direction to divert the return flow out one side of the dump valve to the atmosphere. In the open position, atmospheric air may be drawn through the other side of the dump valve. In the open position, one side wall 17 is disposed diagonally across the flow channel and the other side wall 17 is disposed outwardly of the flow channel.

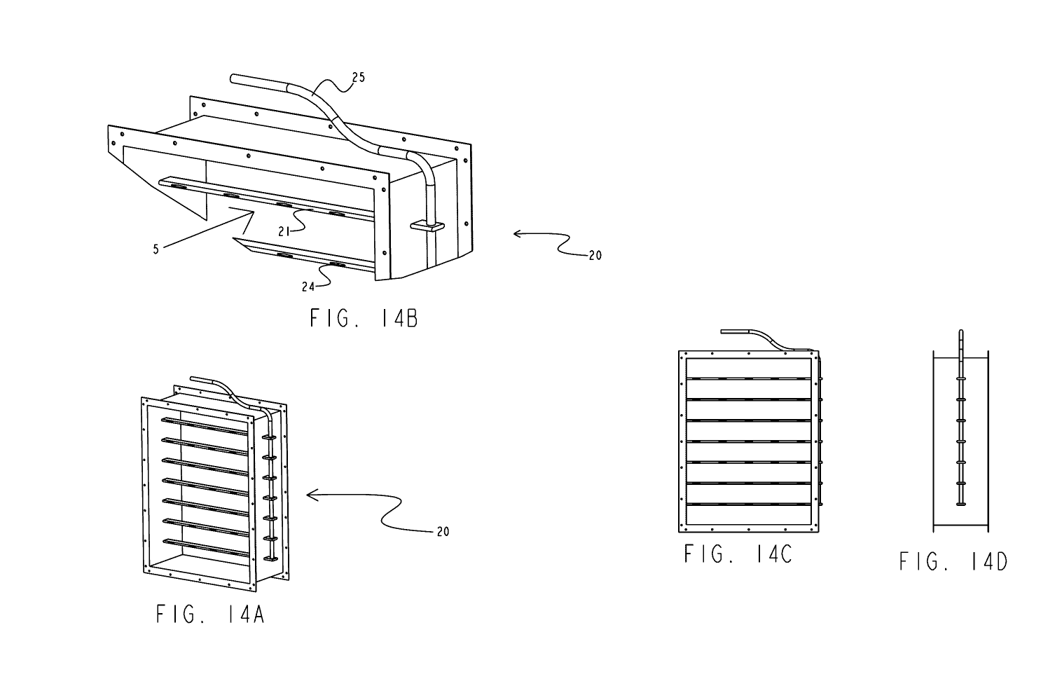

With reference to FIG. 19B, an air sample module 20 may be connected to air handling unit 10, preferably to the upstream side of said unit. With reference to FIGS. 14A-14D, air sample module 20 may comprise at least one sampling reed 21 disposed across the air flow in the module. Sampling reed 21 is hollow with a wall forming an interior chamber, and at least one sampling port 24 through said wall. Port 24 is exposed to the air flow to pass samples into chamber 23. Air sample tube 25 is connected to said reeds to transport samples away for analysis.

The improved capability of the present invention to monitor and to sustain high quality of interior air permits more recirculation of interior air and less introduction atmospheric makeup air. Thus, significant energy expenditures to heat or cool makeup air may be saved.

While the invention has been particularly shown and described with reference to certain embodiments, it will be understood by those skilled in the art that various changes in form and details may be made to the invention without departing from the spirit and scope of the invention as described in the following claims

We claim:

1. An apparatus for supplying air to a room in a building and returning air from the room to a motive source of air circulation, comprising:
supply ductwork for carrying a flow of supply air from the source to the room;
return ductwork for carrying a flow of return air from the room;
at least one supply opening in the supply ductwork, said supply opening at an elevation above the floor of the room and laterally inward from the walls of the room for allowing passage of air from the supply ductwork in a downward direction to the room;
at least one return opening in the return ductwork, said return opening at an elevation above the floor of the room and laterally inward from the walls of the room for allowing passage of air from the room in an upward direction to the return ductwork;
wherein at least one of said at least one return opening is centrally disposed to at least one of said at least one supply opening so that a flow of supply air and flow of return air will cause an air flow pattern within the room in which supply air flows downward from the supply ductwork and return air flows upward to the return ductwork interior of the downward supply flow.

2. The apparatus of claim 1, further comprising
a convergence where supply and return ductwork converge so that return ductwork is disposed interior to supply ductwork for at least a portion of its length downstream of the convergence in the direction of the supply flow.

3. The apparatus of claim 1, further comprising:
a vent comprising an opening in the return ductwork through which return air may enter from the room into the return ductwork, and an opening in the supply ductwork through which supply air may enter the room, wherein the return ductwork opening is centrally disposed to the supply ductwork opening.

4. The apparatus of claim 3, wherein the vent is proximate to the ceiling of the room.

5. The apparatus of claim 3, further comprising a plurality of spaced apart vents.

6. The apparatus of claim 2, wherein:
the supply ductwork comprises main supply ductwork and a plurality of branch supply ducts branching off from the main ductwork; and
the return ductwork comprises main return ductwork and a plurality of branch return ducts branching off from the main return ductwork;
wherein each branch return duct is disposed interior to a branch supply duct; and
said apparatus further comprises a plurality of spaced apart vents, each comprising a first opening to a branch supply duct and a second opening to the branch return duct disposed in said branch supply, said second opening centrally disposed to said first opening.

7. The apparatus of claim 6, further comprising a flow adjuster attached to a branch supply duct, said flow adjuster comprising:
a motorized blade slidable between an open position allowing air flow through the branch supply and return ducts and a closed position blocking said branch ducts.

8. The apparatus of claim 7, wherein:
the blade comprises:
a first blade having a front portion configured to close the branch return duct when in the closed position and a rear portion configured to cooperate with a second blade to close the branch supply duct when in the closed position; and
a second blade with a cutout conforming to the shape of the outside of the branch return duct and configured to cooperate with the first blade to close the branch supply duct when in the closed position.

9. The apparatus of claim 2, wherein the convergence comprises an opening in the side of the supply ductwork and the return ductwork disposed through the opening.

10. The apparatus of claim 9, wherein the opening is formed by a coaxial transition section of the supply ductwork in which:
the longitudinal direction of the supply ductwork changes course to conform with the longitudinal direction of the return ductwork;
the circumference of the supply ductwork expands to accommodate the return ductwork and supply air flow; and
the upstream portion of the transition section channels the supply flow to one side of the return ductwork and the transition section transitions the supply flow into an annular space around the return ductwork.

11. The apparatus of claim 1, further comprising:
a diffuser comprising:
a supply portion configured to direct supply air downward and laterally outward from a supply opening; and
a return portion with at least one opening to allow return air into a return opening centrally disposed to said supply opening.

12. The apparatus of claim 2, further comprising:
a hanger spacer between a supply duct and the return duct disposed within the supply duct.

13. The apparatus of claim 2, further comprising:
a roller spacer between a supply duct and the return duct disposed within the supply duct, said roller spacer comprising:
a body attachable to one of said ducts; and
a wheel attached to the body for rolling against the other of said ducts in the longitudinal direction of the ductwork.

14. The apparatus of claim 1, further comprising:
an air handling unit comprising the motive source of air circulation, said unit comprising a supply side downstream of the source and return side upstream of the source;
the return ductwork connected to the return side of the unit; and
the supply ductwork connected to the supply side of the unit.

15. The apparatus of claim 14 wherein the unit further comprises a dehumidifier.

16. The apparatus of claim 14 wherein the unit further comprises an electrostatic precipitator.

17. The apparatus of claim 14 wherein the unit further comprises an ultraviolet germicidal irradiator.

18. The apparatus of claim 14 wherein the unit further comprises a HEPA filter.

19. The apparatus of claim 14 wherein the unit further comprises a carbon filter.

20. The apparatus of claim 14 wherein the unit further comprises a dump valve.

21. The apparatus of claim 14 wherein the unit further comprises a fresh air intake.

22. The apparatus of claim 14 wherein the unit further comprises an air quality sampler.

23. The apparatus of claim 22, wherein the air quality sampler comprises:
at least one sampling reed;
said at least one sampling reed having an interior chamber, and at least one sampling port through the wall of said chamber;
said at least one sampling port exposed to the return flow allowing air samples to enter the chamber;
an air sample tube connected to said at least one sampling reed for carrying samples away from the reeds.

24. The apparatus of claim 11, wherein the return portion extends lower than the supply portion so that the return air enters the return portion at a lower elevation than where the supply air exits the supply portion.

25. The apparatus of claim 11, wherein the diffuser comprises wireless communications transmitters and receivers.

26. The apparatus of claim 11, wherein the diffuser further comprises a surveillance camera.

27. The apparatus of claim 11, wherein the diffuser further comprises a light.

28. The apparatus of claim 11, wherein the diffuser further comprises one of a smoke detector, carbon dioxide detector and carbon monoxide detector.

29. The apparatus of claim 11, wherein the diffuser further comprises an audio speaker.

30. The apparatus of claim 11, wherein the diffuser further comprises a motion detector.

31. The apparatus of claim 11, wherein the diffuser further comprises an air filter disposed in the return portion.

32. The apparatus of claim 31, further comprising:
a pressure sensor disposed in the return flow downstream of the air filter; and
an indicator light for indicating pressure conditions.

33. The apparatus of claim 11, wherein the diffuser comprises:
circuitry and a circuitry connector adapted for releasable connection with external circuitry.

34. The apparatus of claim 33, wherein the circuitry connector is a pin and socket connector for releasable connection with a compatible connector connected to external circuitry.

35. The apparatus of claim 11, wherein the diffuser comprises a support configured for connection with the ductwork and a cover releasably connected to the support.

36. The apparatus of claim 11, wherein the diffuser comprises a support configured for connection with the ductwork and a cover having a hinged connection to the support so that the cover may swing between an open position and a releasably closed position.

37. The apparatus of claim 1, further comprising a wireway attached to and disposed longitudinally alongside the ductwork, said wireway comprising at least one wire channel for receiving at least one wire.

38. The apparatus of claim 37, wherein the wireway comprises multiple wire channels.

39. The apparatus of claim 38, further comprising a plurality of wires disposed in the wireway.

40. The apparatus of claim 37, wherein the wireway comprises:
a base having at least one wire channel, said channel having a side opening to provide access for sideways installation of a wire into the channel.
a channel cover having a first hinged connection to the base so that the cover may swing between an open position exposing the side opening and a closed position covering the side opening.

41. A method of constructing an apparatus for supplying air to a room in a building and returning air from the room to a motive source of air circulation, comprising:
connecting a first end of supply air ductwork to the downstream side of the motive source;
connecting a first end of return air ductwork to the upstream side of the motive source;
merging the supply air ductwork with the return air ductwork so that the return air ductwork is disposed interior to the supply air ductwork downstream of the merger in the direction of the supply flow;
placing a second end of said supply air ductwork in fluid communication with the air in a room; and
placing a second end of said return air ductwork in fluid communication with the air in said room interior to the second end of said supply ductwork.

42. The method of claim 41 further comprising
installing at least one louver proximate to the second end of said supply air ductwork, said at least one louver configured to direct supply air outwardly of said return portion.

43. A method of providing a flow of circulating air to a room in a building and returning air from the room to the motive source of circulation, comprising:
connecting a first end of supply air ductwork to the downstream side of the motive source for circulating air through the system;
connecting a first end of return air ductwork to the upstream side of said motive source;
placing a second end of said supply air ductwork in fluid communication with the air in a room at an elevation above the floor of the room and laterally inward from the walls of the room;
placing a second end of said return air ductwork in fluid communication with the air in a room interior to the second end of said supply air ductwork at an elevation above the floor of the room and laterally inward from the walls of the room;
activating the motive source to draw air from the room upward through the second end of the return ductwork and force air downward through the second end of the supply air ductwork around the second end of the return ductwork.

44. The method of claim 43, further comprising merging the supply air ductwork with the return air ductwork so that the return air ductwork is disposed interior to the supply air ductwork downstream of the merger in the direction of the supply flow.

45. A method of circulating air in a room in a building and returning air from the room to the motive source of circulation, comprising:
forcing supply air through ductwork into the room through at least one supply opening in the ductwork so as to induce a downward supply current of air in the room below said opening; and
concurrently with said forcing step, drawing return air through ductwork out of the room through at least one return opening in the ductwork located centrally to the at least one supply opening so as to induce a return current of air in an upward direction central to the downward supply current.

46. The method of claim 45, wherein the drawing step comprises drawing return air through ductwork out of the room through an opening vent comprising a supply opening and a return opening located centrally to the supply opening so as to induce a downward supply current and upward return current central to the downward supply current.

47. The method of claim 46, wherein the forcing step and drawing step are performed concurrently through a plurality of opening vents, each vent of said plurality being spaced apart from the other, so as to form a plurality of circulation patterns in the room in which supply air flows downward around an upward flow of return air.

48. The method of claim 47, wherein the drawing step is performed through a return opening that is at a lower elevation than the supply opening.

49. A method of converting an existing supply duct of an air circulation system in a building into a dual duct, comprising:
providing a return duct of small enough cross section to fit into the existing supply duct; and
sliding the return duct inside the supply duct so as to form an annular space between the ducts for carrying a flow of supply air.

50. The method of claim 49, further comprising connecting the portion of the return duct that is furthest upstream in the supply duct flow direction to the upstream side of the motive source of air circulation.
